(19) United States
(12) Patent Application Publication
(10) Pub. No.: US 2004/0104172 A1
(43) Pub. Date: Jun. 3, 2004

(54) Countercurrent chromatography separation of polar sulfonated compounds

(76) Inventors: Adrian Weisz, Silver Spring, MD (US); Yoichiro Ito, Bethesda, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/641,805

(22) Filed: Aug. 15, 2003

Related U.S. Application Data
(60) Provisional application No. 60/405,789, filed on Aug. 23, 2002.
(60) Provisional application No. 60/406,407, filed on Aug. 26, 2002.
(60) Provisional application No. 60/490,313, filed on Jul. 24, 2003.

(51) Int. Cl. B01D 15/08

(52) U.S. Cl. 210/656

(57) ABSTRACT

A method for separating a quantity of a sulfonated polar compound from other compounds in a mixture using countercurrent chromatography is disclosed. Also disclosed are compositions of sulfonated polar compounds in great purity and at high yield.

### RELATED APPLICATIONS

This application claims priority to U.S. Provisional Application Serial No. 60/405,789 filed on Aug. 23, 2002; Serial No. 60/406,407 filed on Aug. 26, 2002; and Serial No. 60/490,313 filed on Jul. 24, 2003. The disclosure of the above-described references is hereby incorporated by reference in its entirety.

### BACKGROUND OF THE INVENTION

1. Field of the Invention

The present invention relates to a method of separating positional isomers of polar compounds using conventional and pH-zone-refining countercurrent chromatography to produce polar compounds in great purity and at high yield.

2. Description of the Related Art

The separation of mixtures of closely related compounds into their constituent chemical species is often very difficult. Although HPLC can be successful for a wide variety of species, it is useful only for very small sample amounts, typically no more than several micrograms of material. Thus, this technique cannot be used for the preparative-scale separation of milligram or gram amounts of purified chemicals.

One known method of compound separation which has been used to isolate larger quantities of material is known as countercurrent chromatography. Countercurrent chromatography (CCC) is a form of liquid-liquid partition chromatography which relies on the continuous contact between two immiscible solvents, one of which is mobile relative to the other, in a flow-through tubular column, free of any solid support matrix. The retention time of a solute in the phase contact region of the system is determined by the volume ratio of the solvents, the partition coefficient of the solute between the solvents, and the degree of contact between the solvents. Like other forms of liquid-liquid partition chromatography, one of the solvents serves as a carrier, drawing the solutes from the other solvent and carrying the solutes out of the column in the order of elution. This carrier solvent is thus referred to as the mobile phase, while the other solvent is referred to as the stationary phase, even though it is not strictly stationary in many applications of the method. Solvent mixing, retention of the stationary phase in the column, and solute partitioning all take place in the column by the aid of a suitable acceleration field established by gravity, centrifugal force or both, and the configuration of the column.

An unusually efficient separation of mixtures of acids or bases can be achieved by one known technique of countercurrent chromatography. In this particular method, the two immiscible liquid solutions which are to serve as the stationary and mobile phases, respectively, are modified prior to the performance of the separation by rendering one of the phases acid and the other basic. Separation of a mixture of acids is then performed in a system in which the acidified solution serves as the stationary phase and the basified solution as the mobile phase. Conversely, separation of a mixture of bases is performed in a system in which the basified solution serves as the stationary phase and the acidified solution as the mobile phase. Individual acid or basic solutes separated by this method elute in contiguous, well-resolved, rectangularly shaped peaks, the solutes eluting in order of their partition coefficients (related to their pKa values and hydrophobicity) and the fractions within any single peak being of substantially constant concentration. In addition to differing partition coefficients, the combined fractions within each peak also differ in pH, successively increasing in the case of a basic mobile phase and successively decreasing in the case of an acidic mobile phase. For this reason, the technique may be referred to for convenience as “pH-zone-refining countercurrent chromatography.” This method is discussed in great detail U.S. Pat. No. 5,332,504 issued Jul. 26, 1994 to Ito et al., the entirety of which is incorporated by reference herein, including any drawings.

Although various CCC techniques have been used to isolate pure chemical species, the method has yet to be developed to its full potential. CCC protocols which can be used to isolate previously difficult or impossible to separate compounds are needed.

### SUMMARY OF THE INVENTION

Disclosed herein are compositions of greater than 99% pure 3-sulfophthalic acid or 4-sulfophthalic acid in a quantity of at least 100 milligrams. Also described are compositions of greater than 99% pure sulfonated polar compounds produced by a method for separating a quantity of a sulfonated polar compound from other compounds in a mixture using countercurrent chromatography. The method comprises charging a cross axis countercurrent chromatographic centrifuge column with a first liquid; introducing the mixture comprising two or more sulfonated polar compounds into a combination of the first liquid and a second liquid, thereby producing a test mixture; introducing the test mixture into the countercurrent chromatographic centrifuge column thus charged with the first liquid; and passing the second liquid through the countercurrent chromatographic centrifuge column to elute the sulfonated polar compound from the countercurrent chromatographic centrifuge column; where the first liquid comprises a solvent that is less polar than water and the second liquid is an aqueous solution.

### BRIEF DESCRIPTION OF THE DRAWINGS

FIG. 1 depicts the preparation of D&C Yellow No. 10 by condensing quinaldine, 1, with phthalic anhydride, 2, and sulfonating the condensation product 3.

FIG. 2 depicts the preparation of 2-(2-quinolinyl)-1H-indene-1,3(2H)-dione-4- or 5-sodium sulfonates (6 or 7, respectively) by condensing quinaldine, 1, with 3- or 4-sulfophthalic anhydride, respectively.

FIGS. 3A and 3B depicts the separation of a mixture of 3-sulfophthalic acid (3-SPA) and 4-sulfophthalic acid (4-SPA) by conventional high-speed countercurrent chromatography (HSCCC). FIG. 3A illustrates the results of HPLC analysis of the original mixture, and FIG. 3B is a high-speed countercurrent chromatogram of the separation of a -230 mg portion of the mixture and HPLC analyses of the separated components.

FIGS. 4A and 4B shows the characterization of the compound contained in fractions 68-76 of the HSCCC separation in FIG. 3 by negative ion ESI mass spectrometry (FIG. 4A), and by ¹H NMR mass spectrometry (in D₂O, 400 MHz) (FIG. 4B).

FIGS. 5A and 5B shows the characterization of the compound contained in fractions 81-91 of the HSCCC separation in FIG. 3 by negative ion ESI mass spectrometry (FIG. 5A), and by ¹H NMR mass spectrometry (in D₂O, 400 MHz) (FIG. 5B).

FIGS. 6A-E depicts the separation of 3-SPA and 4-SPA by conventional and pH-zone-refining HSCCC using the J- and X-type coil planet centrifuges.

### DETAILED DESCRIPTION OF THE PREFERRED EMBODIMENT

Mixtures of highly polar compounds are typically difficult to separate into their constituent chemical species. Although HPLC techniques can sometimes be successful, these are limited to microgram quantities of material and are thus not economical for preparative-scale separation of milligram or gram amounts of highly purified chemical species.

It has been discovered that countercurrent chromatography techniques can be employed to separate different species of polar sulfonated compounds that have resisted isolation in preparative-scale amounts. As described above, countercurrent chromatography is a technique that has been used to separate a variety of compound mixtures, but that has not been previously employed to separate milligram or gram quantities of polar sulfonated compounds. In one embodiment, pH zone countercurrent chromatography has been found especially successful in this application. It has also been found that the use of an X-type coil planet centrifuge (CPC) is beneficial.

For two particular species of polar sulfonated compounds, the use of a cross-axis (X-type) CPC successfully separated preparative quantities (such as 100 mg, gram, or multigram quantities) of material to greater than 99% purity. The cross axis centrifuge facilitated the use of an aqueous mobile phase and thus higher stationary phase retention.

In this embodiment, the techniques were employed successfully to chemical species containing both sulfonic and carboxylic acid groups. In one embodiment, positional isomers of sulfophthalic acids were isolated to better than 99% purity. In another embodiment, over 100 milligram quantities of greater than 99% pure 3-sulfophthalic acid (3-SPA), also known as 3-sulfo-1,2-benzenedicarboxylic acid, and 4-sulfophthalic acid (4-SPA), also known as 4-sulfo-1,2-benzenedicarboxylic acid, were obtained for the first time.

Thus, an aspect of the present invention relates to producing compositions of greater than 99% pure sulfonated polar compounds using a method for separating a quantity of a sulfonated polar compound from other compounds in a mixture using countercurrent chromatography. The method comprises a) charging a cross axis countercurrent chromatographic centrifuge column with a first liquid; b) introducing the mixture comprising two or more sulfonated polar compounds into a combination of the first liquid and a second liquid, thereby producing a test mixture; c) introducing the test mixture into the countercurrent chromatographic centrifuge column thus charged with the first liquid; and d) passing the second liquid through the countercurrent chromatographic centrifuge column to elute the sulfonated polar compound from the countercurrent chromatographic centrifuge column. The first liquid preferably comprises a solvent that is less polar than water and the second liquid is preferably an aqueous solution.

In certain embodiments, the sulfonated polar compound further comprises at least one carboxyl group. In some embodiments, the sulfonated polar compound is a sulfophthalic acid or a salt thereof. In some embodiments, the mixture of compounds to undergo separation comprises a mixture of positional isomers of a sulfophthalic acid or salts thereof.

Salts of compounds can be obtained by reacting the compound with inorganic acids such as hydrochloric acid, hydrobromic acid, sulfuric acid, nitric acid, phosphoric acid, or organic acids, such as methanesulfonic acid, ethanesulfonic acid, p-toluenesulfonic acid, salicylic acid and the like. Salts can also be obtained by reacting the compound with a base to form a salt such as an ammonium salt, an alkali metal salt, such as a sodium or a potassium salt, an alkaline earth metal salt, such as a calcium or a magnesium salt, and the like.

“Positional isomers” are two or more compounds that have identical functional groups but different connectivities. Thus, for example, 2-chlorotoluene and 3-chlorotoluene are positional isomers. The separation method described herein has been found suitable for isolating positional isomers.

In certain embodiments, the separation method of the invention further comprises adding an acid to the test mixture. The acid may be any inorganic acid, such as hydrochloric acid, hydrobromic acid, sulfuric acid, nitric acid, phosphoric acid, or organic acid, such as methanesulfonic acid, ethanesulfonic acid, p-toluenesulfonic acid, salicylic acid and the like. In some embodiments, the acid is hydrochloric acid.

In certain embodiments sufficient acid is added to the test mixture to bring its pH to less than about 6, or less than about 5, or less than about 3, or less than about 2.

In certain embodiments, the methods of the present invention further comprises adding a base to the second liquid. In some embodiments, the base comprises a hydroxide group, which may be selected from the group consisting of sodium hydroxide, potassium hydroxide, lithium hydroxide, calcium hydroxide, magnesium hydroxide, aluminum hydroxide, and ammonium hydroxide.

In certain embodiments sufficient base is added to said second liquid to bring its pH to greater than about 8, greater than about 9, greater than about 10, or greater than about 12. By “about” a certain pH it is meant that the pH of the solution is within a ±0.5 range of the disclosed pH. Thus, for example, a pH of less than about 3 signifies that the pH is less than 3±0.5. In other embodiments, “about less than” a certain pH means that the pH is within a ±0.3, ±0.2, or ±0.1 range of the disclosed pH.

In certain embodiments the first liquid comprises an alcohol. In some of these embodiments the first liquid is purely an alcohol, whereas in other embodiments it is a multi-component liquid, in which at least one of the components is an alcohol. The alcohol may be any alcohol. It may be an alcohol having 1-20 carbon atoms and is preferably of low miscibility with water, i.e., a mixture of the alcohol and water forms a biphasic system. In some embodiments the first liquid is butanol.

In certain embodiments, the first liquid and the second liquid are in the test mixture at a ratio of about 1:1. In other embodiment, the ratio of the two liquids in the test mixture is greater than 1:1, while in still other embodiments, the ratio of the two liquids in the test mixture is greater than 3:1. In yet other embodiments, the ratio of the two liquids in the test mixture is less than 1:1.

In some embodiments the countercurrent chromatography is pH-zone-refining countercurrent chromatography, while in other embodiments the countercurrent chromatography is high speed countercurrent chromatography. Some embodiments of the invention use a J-type HSCCC system while other embodiments use an X-type HSCCC system.

The methods of the present invention are particularly well-suited to purify compounds at preparative scales, such as where the quantity of the pure material obtained is greater than 100 milligrams. Certain combinations are particularly useful in achieving these results. For example, the use of an X-type CPC with a highly polar mobile phase, such as an aqueous solution, allows for a successful separation of highly polar compounds that have very close structural similarities.

In some embodiments, the methods of the present invention are useful in purifying sulfonated compounds that are highly polar. An example of a compound that can be purified is 3-sulfophthalic acid or 4-sulfophthalic acid. Using the methods of the present invention, one can obtain a composition of greater than 99% pure 3-sulfophthalic acid or 4-sulfophthalic acid in a quantity of at least 100 milligrams. No other purification technique known heretofore is capable of achieving these results. In some embodiments, the composition of purified 3-SPA or 4-SPA may have at least 1 gram of the purified compound after running one purification batch.

### Examples

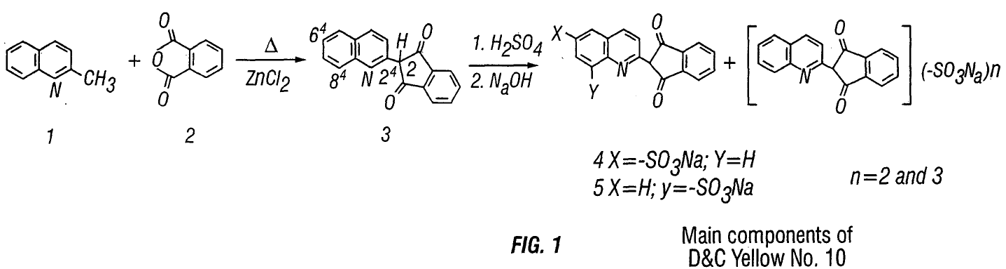

The examples below are non-limiting and depict some of the aspects and embodiments of the invention. D&C Yellow No. 10 (Quinoline Yellow (QY), Colour Index 47005) is a color additive permitted for use in drugs and cosmetics in the USA. It is batch-certified by the U.S. Food and Drug Administration (FDA) to ensure compliance with specifications required by the Code of Federal Regulations (CFR). Code of Federal Regulations, Title 21, Part 74.1710, US Government Printing Office, Washington, D.C., 2001. D&C Yellow No. 10 is manufactured currently as was described for the preparation of QY more than a hundred years ago (E. Jacobsen, U.S. Pat. No. 290,585 (1883)). Specifically, as shown is FIG. 1, one condenses 2-methylquinoline, 1, with phthalic anhydride, 2, and the condensation product, 2-(2-quinolinyl)-1H-indene-1,3(2H)-dione, 3, is then sulfonated. The resulting products are isolated as sodium salts. D&C Yellow No. 10 consists primarily of a mixture of the sodium salts of monosulfonic acid isomers (mainly 4 and 5 in FIG. 1) with up to 15% of the disodium salts of the disulfonated isomers. A variant form of QY contains mostly di- and trisulfonated components and is not certifiable in the United States, but it is used for coloring foods in Europe (E-104) and drugs and cosmetics in Japan (Yellow 203) and other countries.

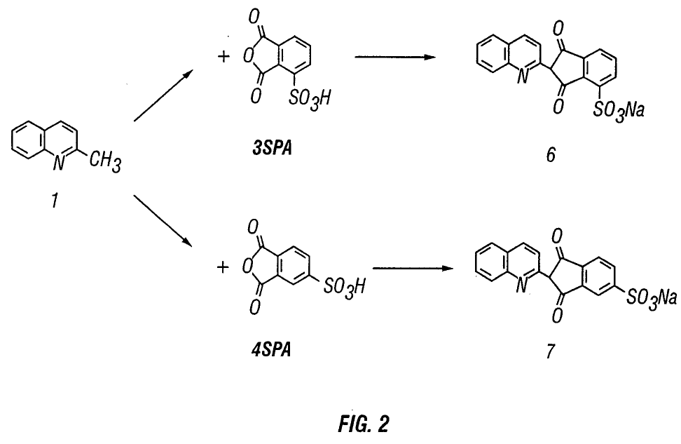

Among the CFR specifications enforced by the FDA are the permitted levels of sulfonated phthalic acids sodium salts (not more than 0.2%) present in D&C Yellow No. 10. These compounds (3-sulfophthalic acid (3SPA), 4-sulfophthalic acid (4SPA) and 3,5-disulfophthalic acid (3,5SPA)) are produced as byproducts during the preparation of D&C Yellow No. 10. Also, their presence in the reaction mixture can result in the formation of adducts sulfonated in the indanedione moiety (e.g., 6 and 7 in FIG. 2).

For the development of analytical methods to be used for FDA batch-certification of D&C Yellow No. 10, purified mono-, di- and trisulfonated components of QY as well as purified sulfophthalic acids are required as reference materials. Most of these compounds are not commercially available, but can be prepared in the laboratory (A. Weisz, E. P. Mazzola, J. E. Matusik, Y. Ito, J. Chromatogr. A 923 (2001) 87; A. Weisz, Y. Ito, in Encyclopedia of Separation Science, Wilson, I. D. (Ed.-in-Chief), vol. 6, Academic Press, London, 2000; 2588-2602). Due to the nonspecific sulfonation of the phthalic acid, 3SPA and 4SPA are obtained as a mixture. This mixture is labeled on commercially-available lots as containing up to 25% 3SPA. While 4SPA may be purchased at a purity of approximately 97%, 3SPA is not commercially available. Besides its use as a reference material, purified 3SPA is needed as the starting material for the preparation of 3,5SPA (K. Lauer, J. Prakt. Chem. 138 (1933) 81), which is also not commercially available. Most of the literature related to these compounds is in patent format and pertains to their use as starting materials for the preparation of sulfonated phthalocyanine dyes or for other applications (K. Sakamoto, E. Ohno-Okumura, Color. Technol. 117 (2001) 82; M. Ganschow, D. Wohrle, G. Schultz-Ekloff, J. Porphyrins Phthalocyanines 3 (1999) 299; S.Takeoka, T. Hara, K. Fukushima, K. Yamamoto, E. Tsuchida, Bull. Chem. Soc. Jpn. 71 (1998) 1471).

While two analytical methods for the separation of 3SPA from 4SPA have been published, (T. Reemtsma, J. Chromatogr. A 919 (2001) 289; G. R. Bear, C. W. Lawley, R. M. Riddle, J. Chromatogr. 302 (1984) 65), no preparative-scale separation method for these isomers has been reported. As described herein, high-speed countercurrent chromatography (HSCCC), previously described by Y. Ito, in: Y. Ito, W. D. Conway (Eds.), High-Speed Countercurrent Chromatography, Wiley, New York, 1996, pp. 3-44, was chosen to separate gram-quantities of a mixture of 3SPA/4SPA that contained approximately 10% 3SPA. Countercurrent chromatography is a liquid-liquid partition technique that does not involve use of a solid support. In conventional HSCCC, one of the liquid phases (the stationary phase) is retained in an Ito multilayered-coil column by centrifugal force while the other liquid phase (the immiscible or aqueous phase) is pumped through the column. The separation depends on the partition coefficient of the solute and the retention of the stationary phase

A variation of HSCCC was relatively recently developed and is known as pH-zone-refining CCC (Y. Ito and A. Weisz, pH-Zone-Refining Countercurrent Chromatography, U.S. Pat. No. 5,332,504, Jul. 26, 1994; Weisz, A., Scher, A. L., Shinomiya, K., Fales, H. M. and Ito, Y., J. Am. Chem. Soc., 116, 704-708 (1994); Y. Ito, in: Y. Ito, W. D. Conway (Eds.), High-Speed Countercurrent Chromatography, Wiley, New York, 1996, pp. 121-175; Y. Ito, Y. Ma, J. Chromatogr. A 753 (1996) 1). pH-Zone-refining CCC enables the separation of organic acids and bases according to their pKa values and hydrophobicities. This technique was used previously for the separation of dyes and intermediates that contain carboxylic or sulfonic acid groups (A. Weisz, E. P. Mazzola, J. E. Matusik, Y. Ito, J. Chromatogr. A 923 (2001) 87; A. Weisz, Y. Ito, in Encyclopedia of Separation Science, Wilson, I. D. (Ed.-in-Chief), vol. 6, Academic Press, London, 2000; 2588-2602; A. Weisz, in: Y. Ito, W. D. Conway (Eds.), High-Speed Countercurrent Chromatography, Wiley, New York, 1996, pp. 337-384). The separations are performed with various types of coil planet centrifuges (CPC) some of which were described earlier in Y. Ito, in: Y. Ito, W. D. Conway (Eds.), High-Speed Countercurrent Chromatography, Wiley, New York, 1996, pp. 3-44. One system that is commercially available is the standard J-type HSCCC system (Y. Ito, CRC Crit. Rev. Anal. Chem., 17 (1986) 65), that provides excellent resolution.

Another system that currently exists as a prototype is the X-type HSCCC system (a cross-axis system), (K. Shinomiya, J.-M. Menet, H. M. Fales, and Y. Ito, J. Chromatogr. 644, (1993) 215-229), that provides higher retention of the stationary phase and is used for separations of peptides, (M. Knight, M. O. Fagarasan, K. Takahashi, A. Z. Geblaoui, Y. Ma, Y. Ito, J. Chromatogr. A 702 (1995) 207-214), and proteins, (Y. Shibusawa, in: Y. Ito, W. D. Conway (Eds.), High-Speed Countercurrent Chromatography, Wiley, New York, 1996, pp. 385-414; Y. Wei, T. Zhang, Y. Ito, J. Chromatogr. A, 917 (2001) 347-351).

As described herein, HSCCC in its two forms, conventional and pH-zone-refining CCC, was applied to the preparative separation of 3SPA and 4SPA from a commercial mixture using the J- and X-type coil planet centrifuges, respectively.

The mixture of 3- and 4-sulfophthalic acids trisodium salts (labeled “4-sulfophthalic acid, trisodium salt, tech., 75%. Remainder 3-sulfophthalic acid, trisodium salt”) used for CCC separations was purchased from Aldrich (Milwaukee, Wis.). n-Butanol, water, hydrochloric acid (approximately 37%), formic acid, ammonium hydroxide (28-30%) and acetonitrile were from J. T. Baker (Philipsburg, N.J.). Phosphoric acid (85%) was from Fisher Scientific (Fair Lawn, N.J.).

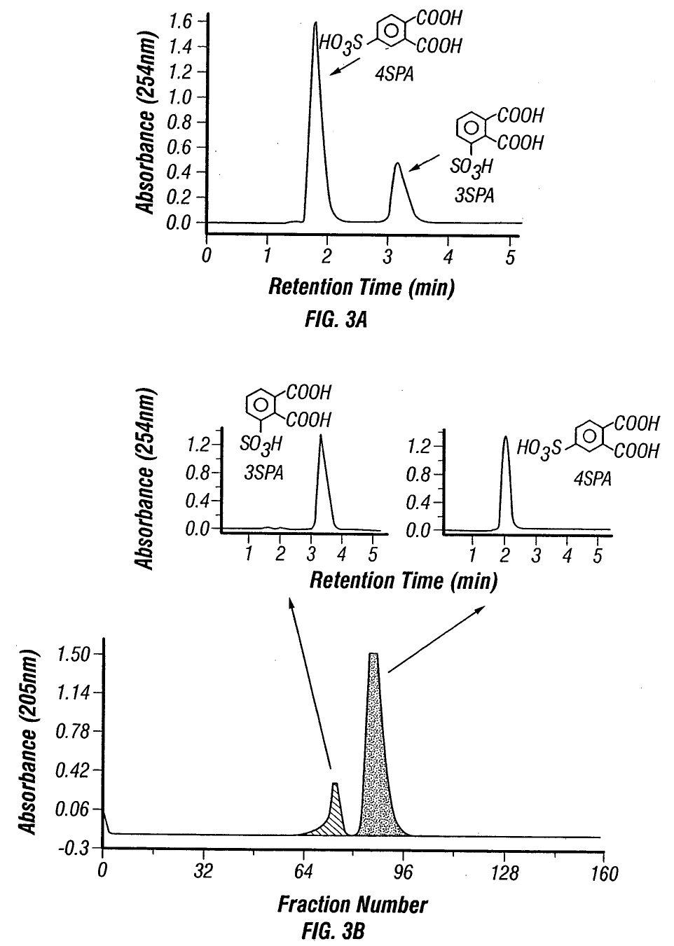

The results of HPLC analysis of the commercial mixture of 3- and 4SPA trisodium salt (labeled “4-sulfophthalic acid, trisodium salt, tech., 75%. Remainder 3-sulfophthalic acid, trisodium salt”) are shown in FIG. 3A. The mixture was found by HPLC (206 nm) to contain approximately 10% 3SPA.

Analytical reversed phase HPLC experiments described herein were performed with a Waters Alliance 2690 Separation Module (Waters, Milford, Mass.). The eluent was 10 mM phosphoric acid (pH approximately 2.4). The column (Prodigy ODS (2), 5 μm particle size, 100×1.0 mm I.D., Phenomenex, Torrance, Calif., USA) was eluted isocratically at 0.1 ml/min. The effluent was monitored with a Waters 996 Photodiode Array Detector set at 254 nm. Injection volume was 5 μl.

A 10 μl aliquot from the CCC collected fractions was diluted with HPLC eluent (0.5 ml) and filtered through a Mini-UniPrep 0.45-μm pore size PTFE syringeless filter device (Whatman, Clifton, N.J.) prior to chromatography. For the 5- and 10 g separations where the collected fractions were more concentrated, 50 μl from the above solution was diluted further by a factor of 10 prior to filtration and injection.

The mass spectra described herein were acquired with a LCQ ion trap mass spectrometer (Finnigan Mat, ThermoQuest, San Jose, Calif., USA). The instrument was fitted with an electrospray (ESI) source. All samples were dissolved in acetonitrile: water (1:1) to which was added 0.1% formic acid, and infused at a rate of 3 μl/min. Lens voltages were optimized in negative ion mode by tuning on the ion of interest. The data was acquired and processed using the Xcalibur software v. 1.0. The negative ion ESI parameters were: sheath gas 60 arbitrary units, spray voltage 4.5 kV, capillary temperature 200° C., capillary voltage 26 V.

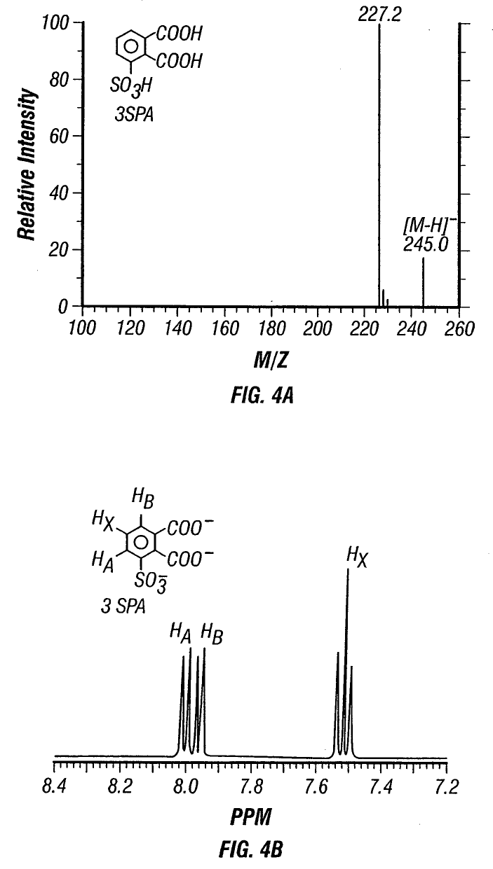

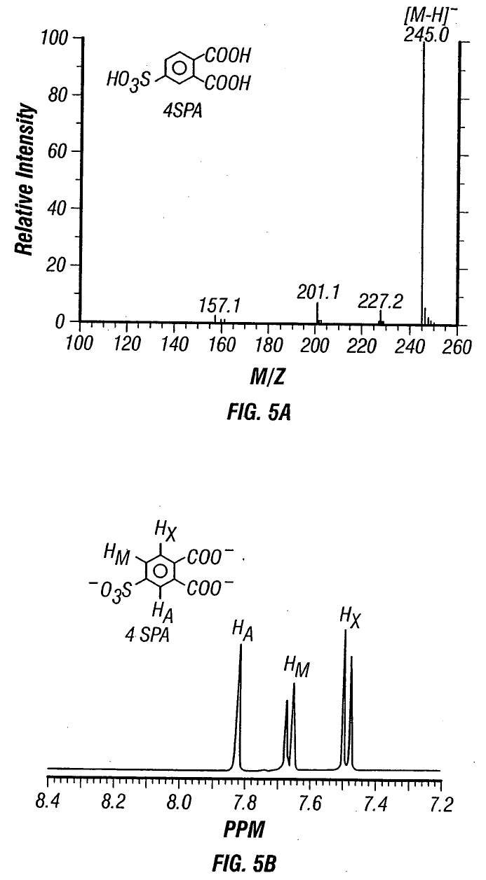

The ¹H-NMR spectra of 3-SPA and 4-SPA described herein were obtained on a Varian VXR-400S spectrometer operating at 400 MHz. Approximately 10 mg each of the purified compounds were dissolved in 140 μl of D₂O and the spectra recorded with standard 10-ppm spectral widths and acquisition parameters. The following signals were obtained and assigned for each of the two isolated sulfophthalic isomers: 3SPA, (3-sulfophthalic acid, FIG. 4), 8.00 ppm (dd, 8,1 Hz; HA), 7.96 ppm (dd, 8,1 Hz; HB) and 7.51 ppm (t, 8 Hz; Hx); 4SPA, (4-sulfophthalic acid, FIG. 5), 7.83 ppm (d, 1.7 Hz; HA), 7.67 ppm (dd, 8, 1.7 Hz; HM) and 7.49 ppm (d, 8 Hz; Hx).

### Example 1

### 3-SPA/4-SPA Separation Using Conventional High-Speed Countercurrent Chromatography

Conventional high-speed countercurrent chromatography (HSCCC) separations were performed with a J-type high-speed CCC system (Model CCC-1000, Pharma-Tech Research, Baltimore, Md., USA) that consisted of a column (three Ito multilayer-coils connected in series made of 1.6 mm I.D. Tefzel tubing with a total capacity of ˜325 ml) mounted on a rotating frame, a speed controller and an LC pump. To facilitate data collection, several improvements were made to this basic system including computerized data acquisition. The instrument used, along with the added improvements, was previously described in A. Weisz, A. L. Scher, Y. Ito, J. Chromatogr. A 732 (1996) 283-290 (incorporated by reference herein in its entirety, including any drawings).

The conventional HSCCC separations were performed following the general directions described earlier in Y. Ito, W. D. Conway (Eds.), High-Speed Countercurrent Chromatography, Wiley, New York, 1996, pp. 3-44 (incorporated by reference herein in its entirety, including any drawings). The solvent system was chosen so that the value of the partition coefficient of the components, K_{upper Phase/Lower Phase}, (KUP/LP) would be in the vicinity of 1. The two-phase solvent system used consisted of n-butanol/water (600 ml:600 ml). To this mixture was added 4.5 ml of concentrated hydrochloric acid (HCl conc.) and the pH became approximately 1.2. The sample in this solvent system had a K_{UP/LP} of 0.7. The solvent system was equilibrated in a separatory funnel, and the two phases were separated before use, resulting in 650 ml of upper organic phase (UP) and 540 ml of lower aqueous phase (LP). The organic UP was used as the stationary phase and the aqueous LP was used as the mobile or aqueous phase.

The separation was initiated by filling the entire column with the first liquid (the stationary phase) using the LC pump, and then loading the test mixture comprising the 3- and 4-SPA sample dissolved in a mixture of the first and second liquids (the stationary and aqueous phases) in the ratio of 1:1, e.g., 5 ml: 5 ml for a 230 mg sample portion. To the test mixture was added HCl conc. until the pH of the test mixture became 0.9. The second liquid (the mobile or aqueous phase) was then pumped into the column at 3 ml/min while the column was rotated at 850 rpm. The column effluent was monitored (UV-scanning from 220 to 450 nm while the adjustable pathlength of the preparative flow cell was set to approximately 0.06 mm) and a fraction collector was used to obtain 3 ml fractions. The fractions collected were brought to dryness using a speed vac concentrator and were analyzed by HPLC.

FIG. 3B shows the countercurrent chromatogram obtained for the separation of 230 mg of this mixture by conventional HSCCC using a commercially-available J-type CPC. The solvent front (first fraction containing the mobile phase) emerged at fraction 58 and the retention of the stationary phase, measured after the separation, was 41.5% of the total column volume. The chromatogram consisted of two peaks. The fractions that corresponded to these peaks (68-76 and 81-91) contained single components, as shown by the associated HPLC chromatograms in FIG. 3B, which were isolated and were identified by negative ion ESI/MS and ¹H NMR as 3SPA (FIG. 4) and 4SPA (FIG. 5), respectively.

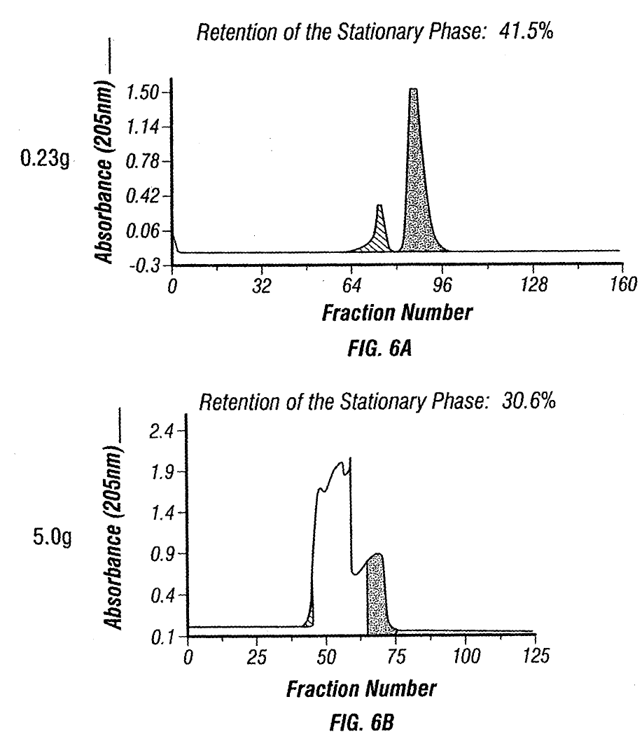

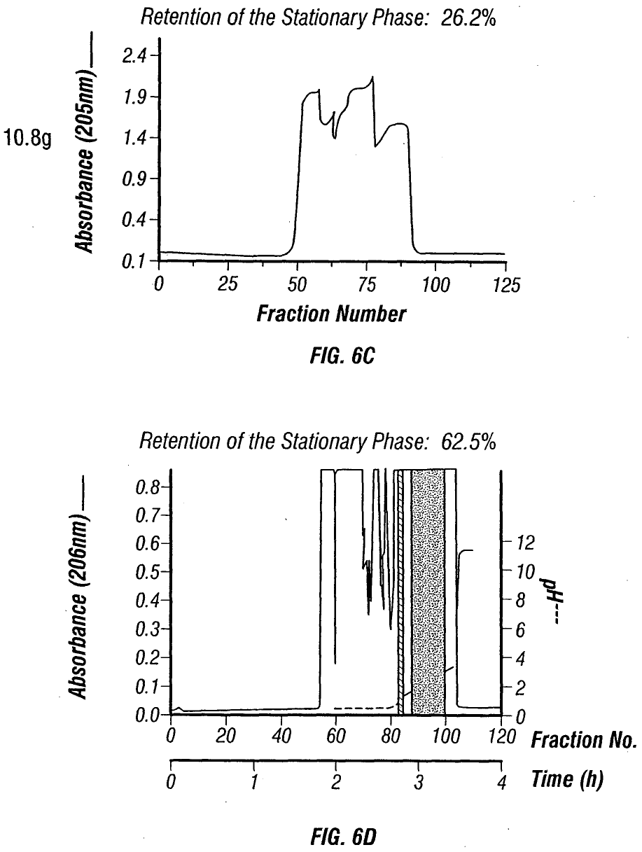

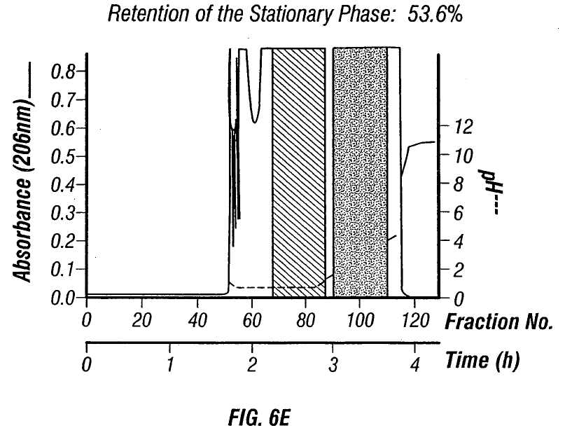

Attempts to separate larger quantities of this mixture, 5 and 10 g portions, by conventional HSCCC failed mainly due to the poor retention of the stationary phase. The retention of the stationary phase (which is a measure for the quality of the separation) dropped to 30.6% and 26.2%, respectively. The CCC chromatograms for these separations are shown in FIGS. 6A-E. The experiment that involved 5 g of sample, resulted in partial separation of the 4SPA while the experiment that involved 10 g of sample, resulted only in fractions with various degrees of mixture.

To separate larger quantities of these isomers, a different approach was necessary. The samples (5 and 10 g) were subjected to pH-zone-refining CCC (a relatively new HSCCC technique for the preparative-scale separation of ionizable compounds) using an X-type (cross-axis) CPC (Y. Ito and A. Weisz, pH-Zone-Refining Countercurrent Chromatography, U.S. Pat. No. 5,332,504, Jul. 26, 1994; Weisz, A., Scher, A. L., Shinomiya, K., Fales, H. M. and Ito, Y., J. Am. Chem. Soc., 116, 704-708 (1994); Y. Ito, in: Y. Ito, W. D. Conway (Eds.), High-Speed Countercurrent Chromatography, Wiley, New York, 1996, pp. 121-175; Y. Ito, Y. Ma, J. Chromatogr. A 753 (1996) 1; Y. Ito, Y. Ma, J. Chromatogr. A 753 (1996) 1 (all of which are incorporated by reference herein in their entirety, including any drawings)). The X-type CPC has a higher capability for retention of the stationary phase than the J-type instrument. This capability was demonstrated when stationary phase retention of 62.5% and 53.6% were obtained for the 5 and 10 g separations, respectively.

### Example 2

### 3-SPA/4-SPA Separation Using pH-Zone-Refining Countercurrent Chromatography

The pH-zone-refining CCC separations were performed with a prototype of an X-type high-speed CCC system (cross-axis CPC) (K. Shinomiya, J.-M. Menet, H. M. Fales, and Y. Ito, J. Chromatogr. 644, (1993) 215-229 (incorporated by reference herein in its entirety, including any drawings)), that consisted of a column (two Ito multilayer-coils connected in series made of 2.6 mm I.D. Teflon tubing with a total capacity of about 575 ml) mounted on a rotating frame with the axis of the column rotation perpendicular to the centrifuge axis, a speed controller and an LC pump. The instrument used was previously described and depicted in a photograph. Id.

The pH-zone-refining CCC separations followed previously established procedures (A. Weisz, Y. Ito, in Encyclopedia of Separation Science, Wilson, I.D. (Ed.-in-Chief), vol. 6, Academic Press, London, 2000; 2588-2602 (incorporated by reference herein in its entirety, including any drawings)). The two-phase solvent system used consisted of n-butanol/water (1:1). The solvent system was equilibrated in a separatory funnel, and the two phases were separated before use. The organic UP was acidified with HCl conc. to pH˜0.5 (488 mM in HCl). The aqueous LP was rendered basic by addition of ammonium hydroxide resulting in a ˜105 mM solution in NH₃ (pH approximately 10.7). The acidic organic phase was used as the stationary phase and the basic LP was used as the mobile or aqueous phase.

The separation was initiated by filling the entire column with the ifrst liquid (the stationary phase) using the LC pump, and then loading the test mixture comprising the 3- and 4-SPA sample dissolved in a mixture of the first and second liquids (the stationary and aqueous phases) in the ratio of 4:1, e.g., 40 ml: 10 ml for a 5 g sample portion. To the test mixture was added HCl conc. until the pH of the test mixture became approximately 0.5. The second liquid, the mobile or aqueous phase, was then pumped into the column at 2 m/min while the column was rotated at 715 rpm in the combined head to tail elution mode P₁-H-O (Y. Ito, in: Y. Ito, W. D. Conway (Eds.), High-Speed Countercurrent Chromatography, Wiley, New York, 1996, pp. 3-44; K. Shinomiya, J.-M. Menet, H. M. Fales, and Y. Ito, J. Chromatogr. 644, (1993) 215-229). The absorbance of the eluate was continuously monitored at 206 nm and 4-ml fractions were collected. The pH of each eluted fraction was measured with a pH meter. The fractions collected were brought to dryness using a speed vac concentrator and were analyzed by HPLC.

The pH-zone-refining CC chromatograms for these separations are shown in FIG. 6. The chromatograms have the broad rectangular shape characteristic of pH-zone-refining CCC. Id. The two broad absorbance plateaus (hatched area, more evident in the 10 g separation) correspond to the two pH plateaus (dotted line). Each plateau represents elution of a pure compound. For the 10 g separation, the eluates collected in fractions 76-88 contained approximately 440 mg of over 99% pure 3-SPA (by HPLC) and the eluates collected in fractions 98-107 contained approximately 4.73 g of over 99% pure 4-SPA (by HPLC).

The embodiments of the invention described above thus produced for the first time greater than 99% pure 3SPA and 4SPA in quantities of greater than 100 mg, and in some embodiments, greater than 1 gram of purified material.

The references alluded to in the text above are incorporated herein in their entirety, including any drawings.

What is claimed is:

1. A composition of greater than 99% pure 3-sulfophthalic acid in a quantity of at least 100 milligrams.

2. The composition of claim 1, wherein said quantity is at least 1 gram.

3. A composition of greater than 99% pure 4-sulfophthalic acid in a quantity of at least 100 milligrams.

4. The composition of claim 3, wherein said quantity is at least 1 gram.

5. A composition of greater than 99% pure sulfonated polar compound produced by a method for separating a quantity of a sulfonated polar compound from other compounds in a mixture using countercurrent chromatography, said method comprising:
a) charging a cross axis countercurrent chromatographic centrifuge column with a first liquid;
b) introducing a mixture comprising two or more sulfonated polar compounds into a combination of said first liquid and a second liquid, thereby producing a test mixture;
c) introducing said test mixture into said column thus charged with said first liquid; and
d) passing said second liquid through said column to elute said sulfonated polar compound from said column;
wherein said first liquid comprises a solvent that is less polar than water and said second liquid is an aqueous solution.

6. The composition of claim 5, wherein said method further comprises rotating said column while said second liquid is passed therethrough.

7. The composition of claim 5, wherein said sulfonated polar compound further comprises at least one carboxyl group.

8. The composition of claim 5, wherein said sulfonated polar compound is a sulfophthalic acid or a salt thereof.

9. The composition of claim 5, wherein said mixture comprises a mixture of positional isomers of a sulfophthalic acid or salts thereof.

10. The composition of claim 5, wherein said method further comprises adding an acid to the test mixture.

11. The composition of claim 10, wherein said acid is HCl.

12. The composition of claim 5, wherein said method further comprises adding sufficient acid to the test mixture to bring its pH to less than about 3.

13. The composition of claim 5, wherein said method further comprises adding a base to said second liquid.

14. The composition of claim 13, wherein said base comprises a hydroxide group.

15. The composition of claim 14, wherein said base is ammonium hydroxide.

16. The composition of claim 5, wherein said method further comprises adding sufficient base to the second liquid to bring its pH to greater than about 9.

17. The composition of claim 5, wherein said first liquid comprises an alcohol.

18. The composition of claim 17, wherein said first liquid is butanol.

19. The composition of claim 5, wherein said first liquid and said second liquid are in said test mixture at a ratio of 1:1.

20. The composition of claim 5, wherein said first liquid and said second liquid are in said test mixture at a ratio of greater than 1:1.

21. The composition of claim 5, wherein said first liquid and said second liquid are in said test mixture at a ratio of greater than 3:1.

22. The composition of claim 5, wherein said first liquid and said second liquid are in said test mixture at a ratio of less than 1:1.

23. The composition of claim 5, wherein said countercurrent chromatography is pH-zone-refining countercurrent chromatography.

24. The composition of claim 5, wherein said quantity is greater than 100 milligrams.

25. The composition of claim 5, wherein said sulfonated polar compound is 3-sulfophthalic acid.

26. The composition of claim 5, wherein said sulfonated polar compound is 4-sulfophthalic acid.

27. The composition of claim 5, wherein the method is performed using an X-type HSCCC system.

28. The composition of claim 5, wherein the method is performed using a J-type HSCCC system.
